SEQUENCE LISTING (44 sequences)

SEQ ID NO: 1 - DNA, 22 bp, synthetic construct
  source 1..22: mol_type = other DNA
tgtttcactgggctggtttagc

SEQ ID NO: 2 - DNA, 24 bp, synthetic construct
  source 1..24: mol_type = other DNA
gagaagtctttcaccatgtcacga

SEQ ID NO: 3 - DNA, 22 bp, synthetic construct
  source 1..22: mol_type = other DNA
tgtttcactgggctggtttagc

SEQ ID NO: 4 - DNA, 24 bp, synthetic construct
  source 1..24: mol_type = other DNA
gagaagtctttcaccatgtcacga

SEQ ID NO: 5 - DNA, 20 bp, synthetic construct
  source 1..20: mol_type = other DNA
gggggctatgaatgcacggt

SEQ ID NO: 6 - DNA, 20 bp, synthetic construct
  source 1..20: mol_type = other DNA
ggcaatgcatcaggcacctc

SEQ ID NO: 7 - DNA, 25 bp, synthetic construct
  source 1..25: mol_type = other DNA
tgtcattcaagatctccttgttcct

SEQ ID NO: 8 - DNA, 25 bp, synthetic construct
  source 1..25: mol_type = other DNA
caaattggatatacctccatctcgc

SEQ ID NO: 9 - DNA, 20 bp, synthetic construct
  source 1..20: mol_type = other DNA
ttgggcttcagctttccctt

SEQ ID NO: 10 - DNA, 20 bp, synthetic construct
  source 1..20: mol_type = other DNA
aactcttgcctctgcctgtc

SEQ ID NO: 11 - DNA, 21 bp, synthetic construct
  source 1..21: mol_type = other DNA
gtccatcatgagtacgcgaca

SEQ ID NO: 12 - DNA, 20 bp, synthetic construct
  source 1..20: mol_type = other DNA
attgcaggaccaatgaccgt

SEQ ID NO: 13 - DNA, 22 bp, synthetic construct
  source 1..22: mol_type = other DNA
tcacagcgggtatgaatttcca

SEQ ID NO: 14 - DNA, 22 bp, synthetic construct
  source 1..22: mol_type = other DNA
actgtgacaactttccccaaca

SEQ ID NO: 15 - DNA, 20 bp, synthetic construct
  source 1..20: mol_type = other DNA
taaaggcctgctgacatgct

SEQ ID NO: 16 - DNA, 20 bp, synthetic construct
  source 1..20: mol_type = other DNA
gattttccagcccttgcagc

SEQ ID NO: 17 - DNA, 20 bp, synthetic construct
  source 1..20: mol_type = other DNA
gcacgcggctaaagttatgg

SEQ ID NO: 18 - DNA, 20 bp, synthetic construct
  source 1..20: mol_type = other DNA
ggccccagctaaaatccctt

SEQ ID NO: 19 - DNA, 20 bp, synthetic construct
  source 1..20: mol_type = other DNA
ggaagccttatggacggcta

SEQ ID NO: 20 - DNA, 20 bp, synthetic construct
  source 1..20: mol_type = other DNA
tggtatgataaccggcaccc

SEQ ID NO: 21 - DNA, 20 bp, synthetic construct
  source 1..20: mol_type = other DNA
ccctagcaagagatggggtg

SEQ ID NO: 22 - DNA, 20 bp, synthetic construct
  source 1..20: mol_type = other DNA
tgggaccacaataccaaggc

SEQ ID NO: 23 - DNA, 20 bp, synthetic construct
  source 1..20: mol_type = other DNA
cgtacaggcaaggcagagaa

SEQ ID NO: 24 - DNA, 20 bp, synthetic construct
  source 1..20: mol_type = other DNA
tctcaccctctgttgttggc

SEQ ID NO: 25 - DNA, 20 bp, synthetic construct
  source 1..20: mol_type = other DNA
ggccaaagccagagcaattt

SEQ ID NO: 26 - DNA, 20 bp, synthetic construct
  source 1..20: mol_type = other DNA
agcaccctttaactgctgga

SEQ ID NO: 27 - DNA, 21 bp, synthetic construct
  source 1..21: mol_type = other DNA
agaaagggcaggatggttgag

SEQ ID NO: 28 - DNA, 20 bp, synthetic construct
  source 1..20: mol_type = other DNA
atacagcaacaggcgagtga

SEQ ID NO: 29 - DNA, 20 bp, synthetic construct
  source 1..20: mol_type = other DNA
ttgcctggttttgttcccct

SEQ ID NO: 30 - DNA, 20 bp, synthetic construct
  source 1..20: mol_type = other DNA
aacaccacaaccagtacgga

SEQ ID NO: 31 - DNA, 21 bp, synthetic construct
  source 1..21: mol_type = other DNA
gagaatggcctgcaaggacta

SEQ ID NO: 32 - DNA, 21 bp, synthetic construct
  source 1..21: mol_type = other DNA
aggcaacaaaagctgaagtgt

SEQ ID NO: 33 - DNA, 20 bp, synthetic construct
  source 1..20: mol_type = other DNA
cggtgttcagaggagccaat

SEQ ID NO: 34 - DNA, 20 bp, synthetic construct
  source 1..20: mol_type = other DNA
gaagcaagcaactttccccc

SEQ ID NO: 35 - DNA, 20 bp, synthetic construct
  source 1..20: mol_type = other DNA
gtattgctaaagctgccgcc

SEQ ID NO: 36 - DNA, 20 bp, synthetic construct
  source 1..20: mol_type = other DNA
ggtgaagcaaactgcccaag

SEQ ID NO: 37 - DNA, 20 bp, synthetic construct
  source 1..20: mol_type = other DNA
actctccccatttgctggtt

SEQ ID NO: 38 - DNA, 21 bp, synthetic construct
  source 1..21: mol_type = other DNA
gtgtgtggtgaaatgcacagg

SEQ ID NO: 39 - DNA, 130 bp, Solanum lycopersicum
  source 1..130: mol_type = other DNA
tggccaagacccactgcaattgcgggaaaaataataatttgctatgctctatttgaggctacacttcagcttttgttgcctggtaaaagggttgaaggaccgatatctcctactggacacaggcctgtct

SEQ ID NO: 40 - DNA, 22 bp, Solanum lycopersicum
  source 1..22: mol_type = genomic DNA
tggccaagacacaggcctgtct

SEQ ID NO: 41 - DNA, 22 bp, Solanum lycopersicum
  source 1..22: mol_type = genomic DNA
tggccaagacacaggcctgtct

SEQ ID NO: 42 - DNA, 22 bp, Solanum lycopersicum
  source 1..22: mol_type = genomic DNA
tggccaagacacaggcctgtct

SEQ ID NO: 43 - DNA, 131 bp, Solanum lycopersicum
  source 1..131: mol_type = genomic DNA
tggccaagacccactgcaattgcgggaaaaataataatttgctatgctctatttgaggctacacttcagcttttgttgcctggtaaaagggttgaaggaccgatatctcctactggatcacaggcctgtct

SEQ ID NO: 44 - DNA, 129 bp, Solanum lycopersicum
  source 1..129: mol_type = genomic DNA
tggccaacccactgcaattgcgggaaaaataataatttgctatgctctatttgaggctacacttcagcttttgttgcctggtaaaagggttgaaggaccgatatctcctactggagcacaggcctgtct

(19) United States
(12) Patent Application Publication
(10) Pub. No.: US 2024/0384281 A1
(43) Pub. Date: Nov. 21, 2024

(54) METHODS OF IMPROVING VITAMIN D LEVELS IN PLANTS

(71) Applicant: JOHN INNES CENTRE, Norwich (GB)

(72) Inventors: Cathie Martin, Norwich (GB); Jie Li, Norwich (GB)

(21) Appl. No.: 18/689,925

(22) PCT Filed: Sep. 12, 2022
(86) PCT No.: PCT/EP2022/075312

§ 371 Date: Mar. 7, 2024

(30) Foreign Application Priority Data
Sep. 14, 2021 (GB) 2113075.2

(51) Int. Cl. C12N 15/82; A01H 3/02

(52) U.S. Cl. CPC C12N 15/8218; A01H 3/02; C12N 15/8243; C12Y 103/01021

(57) ABSTRACT

Methods for increasing levels of provitamin D3 and/or vitamin D3 in plants are described. Tomato plants were genetical modified resulting in decreased activity of 7-dehydrocholesterol reductase.

### FIELD OF THE INVENTION

The present invention relates to methods for improving levels of vitamin D and/or of provitamin D in plants. The invention also relates to plants obtained by the method, as well as the fruits thereof, and foodstuffs prepared from the plants of the invention.

### BACKGROUND OF THE INVENTION

Vitamin D was identified by its ability to prevent deficiency diseases affecting skeletal development particularly rickets in children, osteomalacia and osteoporosis in adults¹. Vitamin D is converted by two hydroxylation reactions, to products with steroid hormone bioactivities which function not only in calcium homeostasis but also signalling in multiple organs including the heart, the bones, the lungs, intestines, mammary glands and the brain². Consequently, deficiencies in vitamin D impact immune function and inflammation and are associated with increased risk of cancer, particularly breast and colon cancer^{3,4}, Parkinson's Disease⁵, depression⁶, neurocognitive decline⁷, dementia⁸ and, most recently, with the severity of infection by COVID-19⁹. Vitamin D can be synthesised by humans from 7-dehydrocholesterol (7-DHC), following exposure of skin to UV-B light¹⁰, but the major source is dietary¹¹. It has been estimated that approximately 1 billion people world-wide suffer from vitamin D insufficiency¹² and numbers are increasing largely because of inadequate dietary availability. Poor vitamin D status is a major public health problem, in all age groups. The European Food Safety Authority has defined an adequate intake of 15 μg per day for healthy individuals over one year of age, and in the USA the National Institutes of Health recommend 15 μg per day for children and adults rising to 20 μg day for adults over 70 years old. Generally, these intakes cannot be achieved from food sources without supplementation either through fortified foods or through vitamin D supplements. Because humans can synthesise vitamin D₃ themselves from 7-DHC following exposure to UV-B irradiation, most advice for correcting vitamin D₃ insufficiency is based on increasing exposure to sunlight. However, even in individuals with adequate exposure to sunlight, insufficiency in vitamin D₃ is common, due to declining levels of provitamin D₃ in the skin in the elderly (>70 years old), high skin melanin content and burn scar tissue which reduce UV-B penetrance, and intestinal malabsorption syndromes, such as Crohn's Disease and other intrinsic factors.

In addition, the practice of sunbathing has decreased due to concerns about exposure to UV-B irradiation causing skin cancers, and has been accompanied by use of higher protection, UV-blocking sunscreens in those that do sunbathe, which limit vitamin D production.

Although Vitamin D₂ was originally identified in plants, this was eventually shown to be due to fungal infection¹³. Provitamin D₃ (7-DHC) is synthesised by some plants like tomato, on route to cholesterol and steroidal glycoalkaloid (SGA) synthesis, predominantly in leaves. UV-B exposure of leaves of tomato produces vitamin D₃ but, generally, plants are considered to be relatively poor dietary sources, with the best sources being fish and dairy products. Mushrooms and yeast can be used as sources of vitamin D₂, following exposure to UV-B light, but Vitamin D₂ has been reported to be significantly less bioeffective than vitamin D₃ in several epidemiological studies^{14,15}. The increasing popularity of veganism means that an increasing proportion of diets are likely to be vitamin D deficient without additional supplementation, which would need to be vitamin D₂ from mushrooms.

It would be beneficial to provide plants with increased levels of vitamin D and/or of provitamin D.

### SUMMARY OF THE INVENTION

We have engineered the accumulation of provitamin D₃ in tomato by genome editing, taking advantage of a partial duplication of the phytosterol biosynthetic pathway in Solanaceous plants, to provide a biofortified food with the option of supplement production from waste material.

In a first aspect of the invention there is provided a method of improving provitamin D₃ levels in a plant, the method comprising reducing activity of 7-dehydrocholesterol reductase (7-DR) in the plant. In preferred embodiments, the plant is one having a duplication of the 7-DR gene; here, preferably one of the loci is targeted for reduction of activity. As described herein, the 7-DR2 enzyme converts provitamin D_{3/}7-DHC to cholesterol for the synthesis of tomatine in leaves and fruit of the tomato plant. Consequently, inhibiting the activity of 7-DR2 in tomato could result in the accumulation of 7-DHC without any impact on phytosterol and brassinosteroid biosynthesis. The gene and enzyme are referred to herein as SI7-DR2 (that is, the tomato-specific gene), although it will be understood that this is intended to encompass homologs and orthologs in other plant species.

“Reducing activity” may mean reducing or abolishing enzyme activity or expression. In a preferred embodiment, the method comprises introducing a loss of function mutation into at least one copy of the 7-DR gene (preferably SI7-DR2 gene). The mutation may be in the coding sequence. The mutation may be an insertion, deletion, or alteration. In embodiments loss of function mutations may be introduced into multiple copies of the SI7-DR2 gene. Preferably, the mutation is introduced by genome editing, preferably ZFNs, TALENs or CRISPR. In some embodiments, mutagens (for example, radiation) may be used to introduce the mutation. In some embodiments, mutations may be introduced into at least one copy of the gene, and conventional breeding techniques used to generate plants with multiple mutations in the genome, eg, homozygous plants.

In other embodiments, post-transcriptional techniques may be used to reduce or abolish enzyme activity. For example, RNAi, CRISPRi, or antisense techniques can all be used to reduce enzyme levels. The method may comprise introducing an siRNA or antisense molecule into the plant; or may comprise introducing a nucleic acid sequence which encodes an siRNA or antisense molecule into the plant. Such nucleic acid sequence may be stably incorporated into the plant genome.

Where enzyme activity is reduced (but not abolished), the activity is preferably reduced by at least 5%, 10%, 20%, 30%, 40%, 50%, 60%, 70%, 80% or 90% or more compared to the level in a wild-type or control plant. Methods for determining enzyme activity are within the expertise of the skilled person.

In embodiments, the levels of 7-DHC in the plant are increased by at least 5%, 10%, 20%, 30%, 40%, 50%, 60%, 70%, 80% or 90% or more compared to the level in a wild-type or control plant.

The method may further comprise exposing the plant or a part of the plant to UVB radiation. Such exposure is preferably for a time and at an intensity sufficient to convert at least some 7-DHC present in the plant to vitamin D₃. In such embodiments, the levels of vitamin D₃ are increased by at least 5%, 10%, 20%, 30%, 40%, 50%, 60%, 70%, 80% or 90% or more compared to the level in a wild-type or control plant.

In some embodiments, the plant further carries a mutation which increases the penetration of UV-B light into fruit. For example, the y mutation of tomato causes the loss of UV-protecting flavonols from the skin of ‘pink tomatoes’, and so permits further penetration. The method may comprise introducing such a mutation into the plant of the invention; this may be by conventional breeding techniques, or by targeted genome editing. In other embodiments, the methods of the invention may be applied to plants which already carry such mutations; that is, the mutant plant is used as background for reduction of the SI7-DR2 activity.

In one embodiment the plant further carries one or more mutations affecting chlorophyll breakdown in ripe fruit. An example is mutation of the staygreen locus of tomato. Such mutations may help to further elevate levels of 7-DHC in ripe fruit. Stacking of these traits could be achieved by genome editing using ZFNs, TALENs or CRISPR or by introgression.

In one embodiment, the method further comprises processing the plant or a part of the plant to obtain 7-DHC and/or vitamin D₃. Such embodiments may be particularly useful when the part of the plant is not typically consumed; for example, the leaves or stalks of a tomato plant after the tomatoes have been harvested.

In another aspect of the invention, there is provided a genetically altered plant, part thereof or plant cell, wherein the plant, part thereof or plant cell has reduced activity of 7-dehydrocholesterol reductase. The plant, part thereof or plant cell may comprise a loss of function mutation in the 7-DR gene (preferably SI7-DR2 gene).

The plant part may be a seed, fruit, root, tuber, leaf, flower.

In each of the above aspects, the plant is preferably a member of the Solanaceae, more preferably Solanum spp. The plant may be selected from tomato (Solanum lycopersicum), potato (Solanum tuberosum), eggplant (Solanum melongena). In other embodiments, the plant may be Capsicum spp, for example, C. annuum, C. baccatum, C. chinense, C. frutescensand C. pubescens (these including bell peppers and chili peppers). In yet other embodiments, the plant may be Physalis spp, for example, tomatillos.

In another aspect of the invention there is also provided a plant or plant progeny obtained or obtainable by any of the methods described above. In another aspect, there is provided pollen, propagule, progeny, or part of the plant derived from the plant described above wherein said pollen, propagule, progeny, or part comprise a loss of function mutation in the 7-DR gene (preferably SI7-DR2 gene).

In a yet further aspect of the invention, there is provided a food product produced from a plant or plant part of the invention.

Some research indicates that supplementation of diets with vitamin D or provitamin D can have a beneficial effect on reducing serum total cholesterol, LDL cholesterol, and triglyceride levels. The present invention therefore provides a potential route to reduction of cholesterol levels in human or animal subjects in need thereof, by consumption of plants of the invention, or of foodstuffs prepared therefrom. In one aspect, the invention provides a method of reducing cholesterol levels in a human or animal subject in need thereof, the method comprising consuming a plant or foodstuff as herein described. Also provided is a plant or foodstuff as herein described, for use in reducing cholesterol levels in a human or animal subject.

### DESCRIPTION OF THE FIGURES

FIG. 1 shows accumulation of 7-DHC in SI7-DR2 homozygous knock-out lines.

a, The cholesterogenesis pathway (depicted in light green) and phytosterol biosynthesis 15 pathway (depicted in light orange) in tomato, redrawn from Sonawane et al. 7-DHC is converted by 7-DR2 to cholesterol, which can be converted to vitamin D₃ by exposure to UVB light. SMO, C-4 sterol methyl oxidase; C5-SD1, sterol C-5 (6) desaturase 1. b, Five independent SI7-DR2-knockout lines were generated by genome editing. Top: schematic structure of SI7-DR2 gene, with exons indicated as grey arrows. Bottom: recovered mutations in each line are highlighted in light blue. The CRISPR-Cas9-targeted sequences and the protospacer-adjacent motif sequences are shown in blue and red, respectively. c, 7-DHC contents in wild-type (WT) and SI7-DR2-knockout tomato fruit at different stages of ripening (IMG, immature green; MG, mature green; Breaker, fruit turning ripe; B+7, 7 days after breaker-ripe fruit). Data are presented as the mean±s.e.m. From left to right: n=14, 19, 16, 15, 16, 14, 13, 11, 18, 13, 10, 15, 15, 14, 17, 11, 11, 15, 9, 17, 14, 17, 15 and 15 biologically independent fruit samples. ND, not detected. d, 7-DHC content of leaves of wild-type and SI7-DR2-knockout lines. Data are presented as mean±s.e.m. From left to right: n=4, 5, 5, 4, 5 and 4 biologically independent leaf samples. Statistical significance between WT and mutants at each fruit 30 ripening stage (c) or in leaves (d) was assessed using two-tailed t-tests (*P≤0.05, **P≤0.01, ***P≤0.001, ****P≤0.0001).

FIG. 2 shows localisation and quantitative comparison of SGAs and cholesterol in WT and SI7-DR2 knock-out mutant lines and conversion of 7-DHC in SI7-DR2 knock-outs to vitamin D₃ by UV-B irradiation.

a, MALDI images of 7-DHC (m/z 367.33) and its laser-induced derivative ion (m/z 365.32), cholesterol (m/z 369.35) and α-tomatine (m/z 1,034.55). Scale bar, 2 mm. The HotMetal2 colour scale indicates the range of total ion current-normalized intensity. The same metabolite is shown with identical scale intensity for wild-type and mutant samples. It is not straightforward to compare the relative abundance of different metabolites using MALDI images due to potentially different ionization efficiencies. b, α-Tomatine contents of leaves of wild-type and SI7-DR2-knockout lines (mean±s.e.m, n=3 biologically independent leaf samples for each line). c, Relative esculeoside A content of red-ripe (seven days after breaker) fruit of wild-type and SI7-DR2-knockout lines (mean±s.e.m). From left to right: n =6, 6, 5, 8, 10 and 10 biologically independent fruit samples. d, Cholesterol content of leaves of wild-type and SI7-DR2-knockout lines (mean±s.e.m). From left to right, n=4, 5, 5, 4, 5 and 4 biologically independent leaf samples. e, Contents of 7-DHC and vitamin D₃ in control and UVB-treated leaves or fruit (mean±s.e.m, n=4 biologically independent leaf or fruit samples at each stage for control and MUT #2). Tissues of Mut #2 were irradiated by UVB light for 1 h. The experiment was repeated three times. ND, not detected. Statistical significance between WT and mutant values (b-d) and between control and UVB-treated tissue (e) was assessed using two-tailed t-tests (*P≤0.05, **P≤0.01, ***P≤0.001, ****P≤0.0001).

FIG. 3 shows comparisons of WT and SI7-DR2 knock-out plants

a Adult plants of wild type and SI7-DR2 mutants. Scale bar, 20 cm. b Stigmasterol content of leaves from wild type and SI7-DR2 knock out lines (mean±s.e.m, n=3). Statistical significance between WT and mutants was assessed using two-tailed t-tests. No significant difference was detected. See source data for P values where relevant. c MALDI images of 7-dehydrocholesterol (m/z 367.33) and its laser-induced derivative ions (m/z 365.32, m/z 363.31), cholesterol (m/z 369.35) and α-tomatine (m/z 1034.55). Scale bar, 2 mm. The HotMetal2 colour scale indicates the range of total ion current (TIC)-normalised intensity. The same metabolite is shown with identical scale intensity for wild type and mutant samples. More details can be found in online method. d α-tomatine content of immature green fruit of wild type and SI7-DR2 knock out lines (mean ±s.e.m, n=3). Statistical significance between WT and mutants was assessed using two-tailed t-tests (*P≤0.05, **P≤0.01). See source data for P values where relevant. e Cholesterol contents of wild type (WT) and SI7-DR2 knock out tomato fruit during fruit ripening (IMG, immature green; MG, mature green; B, breaker; B+7, seven days after breaker) (mean±s.e.m). From left to right, n=14, 19, 16, 15, 16, 14, 13, 11, 18, 13, 10, 15, 15, 14, 17, 11, 11, 15, 9, 17, 14, 17, 15, and 15. Statistical significance between WT and mutants was assessed using two-tailed t-tests (*P≤0.05, **P≤0.01, ***P≤0.001, ****P≤0.0001). See source data for P values where relevant

FIG. 4 shows further comparisons of WT and SI7-DR2 knock-out plants

a Relative expression levels of genes in the cholesterol and phytosterol biosynthetic pathways in leaves of wild type and SI7-DR2 mutants (mean±s.e.m). SIActin was used as an internal standard. WT, n=5; SI7-DR2 KO, n=15 (combined of 5 samples from each Mut #1, Mut #2 and Mut #3, which carry the same mutation in SI7-DR2). b Relative expression levels of SIC5-SD1 in leaves of wild type and SI7-DR2 mutants. SIActin was used as an internal standard (mean±s.e.m, n=5). Statistical significance between WT and mutants was assessed using two-tailed t-tests (*P≤0.05, **P≤0.01). See source data for P values where relevant. c Representative LC-MS spectra of 7-dehydrocholesterol (7-DHC), vitamin D3 and cholesterol from analysed samples and corresponding authentic standards. d Overlayed chromatogram of extracts from Mut #1 leaf tissues with and without 2 h UV irradiation (indicated as light blue and black, respectively), as well as standard mix (10 μM of 7-DHC, vitamin D3 and cholesterol), m/z=385.3442-385.3480. Vitamin D3 was largely produced in the UV irradiated sample.

### DETAILED DESCRIPTION OF THE INVENTION

The present invention will now be described more fully. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

For the purposes of the invention, a “genetically altered” or “mutant” plant is a plant that has been genetically altered compared to the naturally occurring wild type (WT) plant. In one embodiment, a mutant plant is a plant that has been altered compared to the naturally occurring wild type (WT) plant using a mutagenesis method, such as the mutagenesis methods described herein. In one embodiment, the mutagenesis method is targeted genome modification or genome editing. Targeted genome modification or targeted genome editing is a genome engineering technique that uses targeted DNA double-strand breaks (DSBs) to stimulate genome editing through homologous recombination (HR)-mediated recombination events. In one embodiment, the mutation is introduced using ZFNs, TALENs or CRISPR. In a preferred embodiment, the targeted genome editing technique is CRISPR. The use of this technology in genome editing is well described in the art, for example in U.S. Pat. No. 8,697,359 and references cited herein.

In another embodiment, conventional mutagenesis techniques, such as T-DNA insertional mutagenesis or any known physical or chemical mutagen can be used disrupt genes described herein. In a further example, the expression of one or more genes can be reduced at the level of transcription or translation using gene silencing methods known to the skilled person, such as, but not limited to, the use of small interfering nucleic acids (siNAs) against one or more genes. For example, the siNA may include short interfering RNA (siRNA), double-stranded RNA (dsRNA), micro-RNA (miRNA), antagomirs and short hairpin RNA (shRNA) capable of mediating RNA interference.

In one example, mutations can be used to knock down or knock out expression of the native 7-DR gene (preferably SI7-DR2 gene). Therefore, in this example, the production of 7-DHC in plants is conferred by the presence of an altered plant genome and is not conferred by the presence of transgenes expressed in the plant. In other words, the genetically altered plant can be described as transgene-free. Nonetheless, in an alternative embodiment, the genetically altered plant may be a transgenic plant.

The term “plant” as used herein encompasses whole plants, progeny of the plants, and plant parts, including seeds, fruit, shoots, stems, leaves, roots (including tubers), flowers, tissues and organs. The term “plant” also encompasses plant cells, suspension cultures, callus tissue, embryos, meristematic regions, gametophytes, sporophytes, pollen and microspores.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. The invention also relates to a product derived from a plant as described herein or from a part thereof, more preferably a food product.

In a most preferred embodiment, the plant part or harvestable product is the fruit. Therefore, in a further aspect of the invention, there is provided fruit produced from a plant as described herein.

In an alternative embodiment, the plant part is pollen, a propagule or progeny of the genetically altered plant described herein. Accordingly, in a further aspect of the invention there is provided pollen, a propagule or progeny produced from a plant as described herein.

A control plant as used herein according to all of the aspects of the invention is a plant which has not been modified according to the methods of the invention. In one embodiment, the control plant is a wild type plant. The control plant is typically of the same plant species, preferably having the same genetic background as the modified plant.

Where reference is made herein to “SI7-DR2” (eg, the SI7-DR2 gene or the SI7-DR2 enzyme), this is intended to encompass not only the specific tomato isoform described herein, but also homologs and orthologs in other plants. The skilled person would understand that suitable homologs can be identified by sequence comparisons and identifications of conserved domains. There are predictors in the art that can be used to identify such sequences. The function of the homolog can be identified using methods known in the art. Homologous positions can thus be determined by performing sequence alignments once the homologous sequence has been identified. Thus, the nucleotide sequences described herein can also be applied in performing the invention in other plants.

The terms “introduction”, “transfection” or “transformation” as referred to herein encompass the transfer of an exogenous polynucleotide or construct (such as a nucleic acid construct or a genome editing construct as described herein) into a host cell, irrespective of the method used for transfer. Plant tissue capable of subsequent clonal propagation, whether by organogenesis or embryogenesis, may be transformed with a genetic construct and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

Transformation of plants is now a routine technique in many species. Any of several transformation methods known to the skilled person may be used to introduce one or more genome editing constructs of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems), lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants can also be produced via Agrobacterium tumefaciens mediated transformation.

Optionally, to select transformed plants, the plant material obtained in the transformation is, as a rule, subjected to selective conditions so that transformed plants can be distinguished from untransformed plants. For example, seeds can be planted and, after an initial growing period, subjected to suitable selection by spraying. A further possibility is growing the seeds, if appropriate after sterilization, on agar plates using a suitable selection agent so that only the transformed seeds can grow into plants. Alternatively, no selection is performed, and seeds are planted and grown and SI7-DR2 activity levels or 7-DHC levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of a mutation of interest, copy number and/or genomic organisation. Alternatively or additionally, integration and expression levels of the newly introduced DNA may be monitored using Southern, Northern and/or Western analysis, all techniques being well known to persons having ordinary skill in the art.

The method may further comprise selecting one or more mutated plants, preferably for further propagation. The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

### EXAMPLES

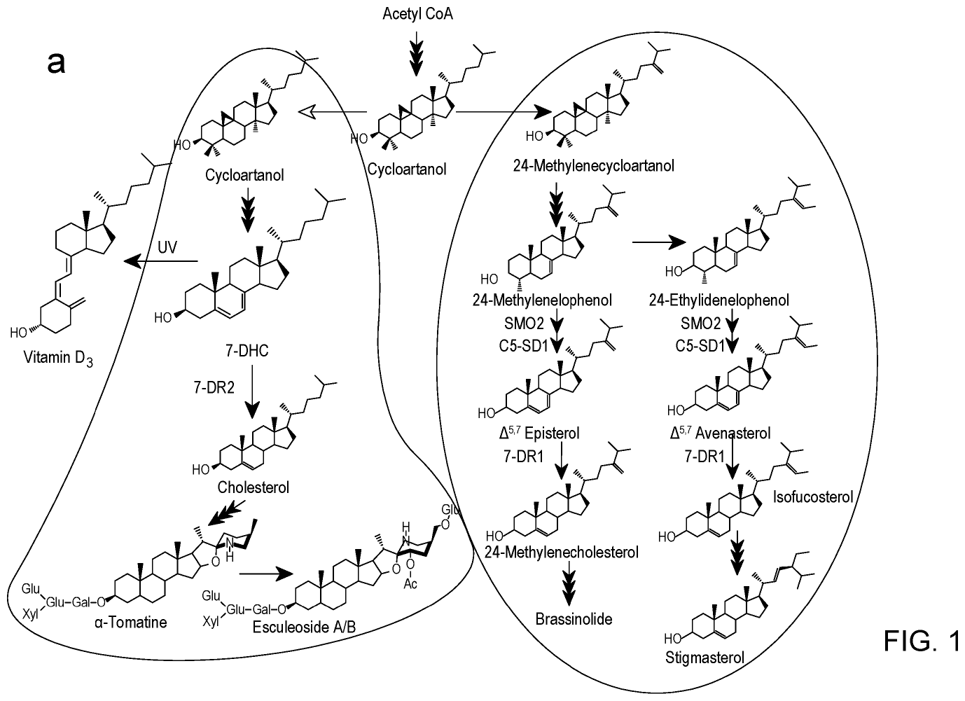

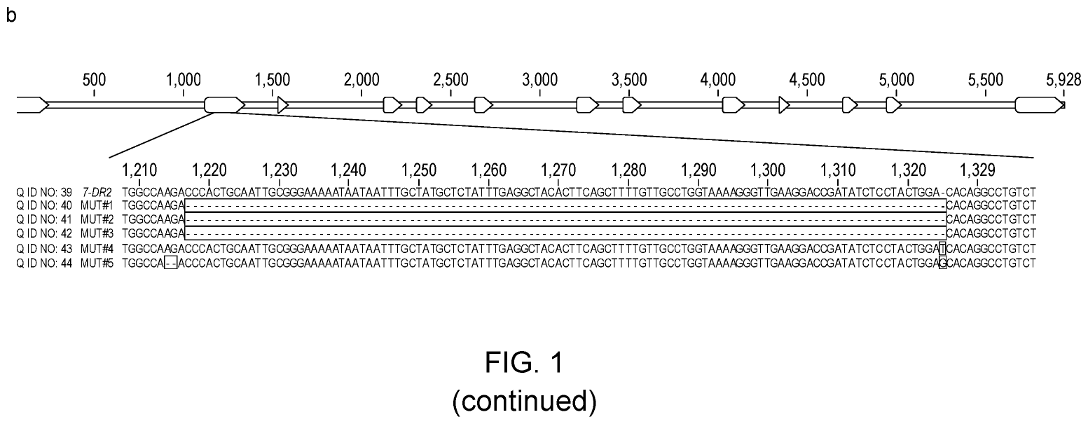

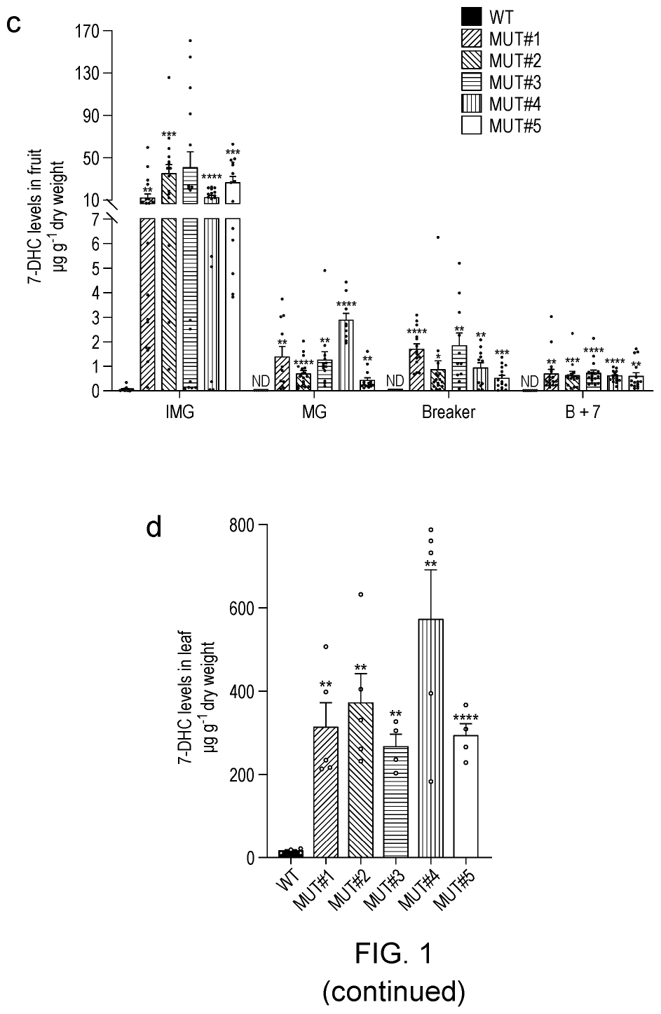

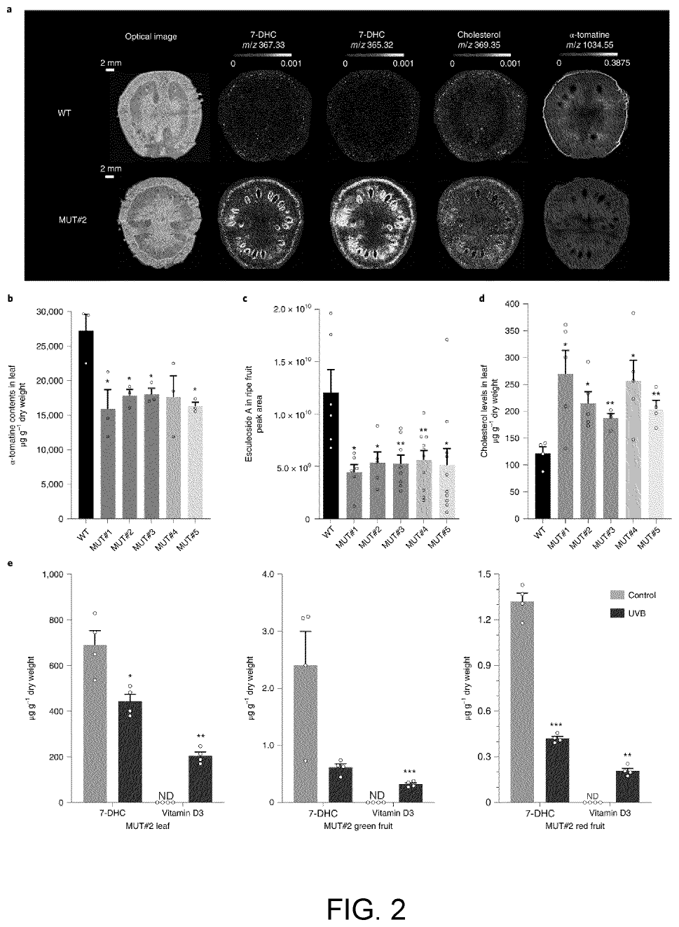

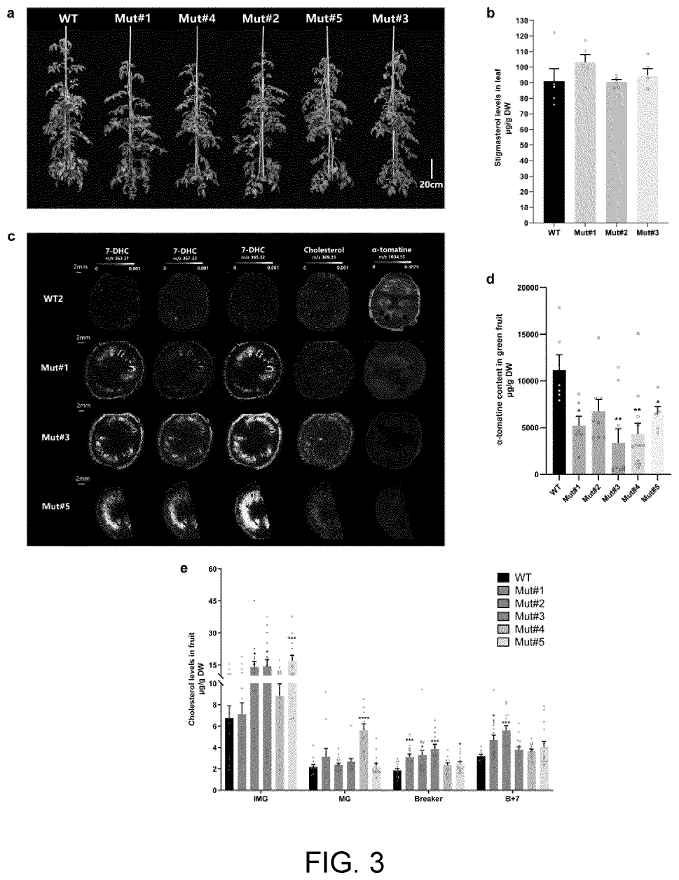

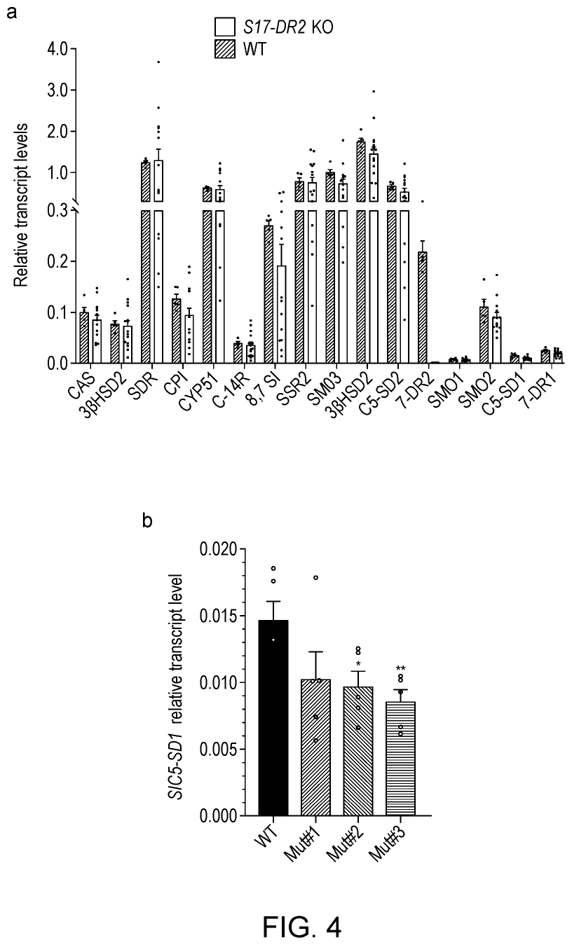

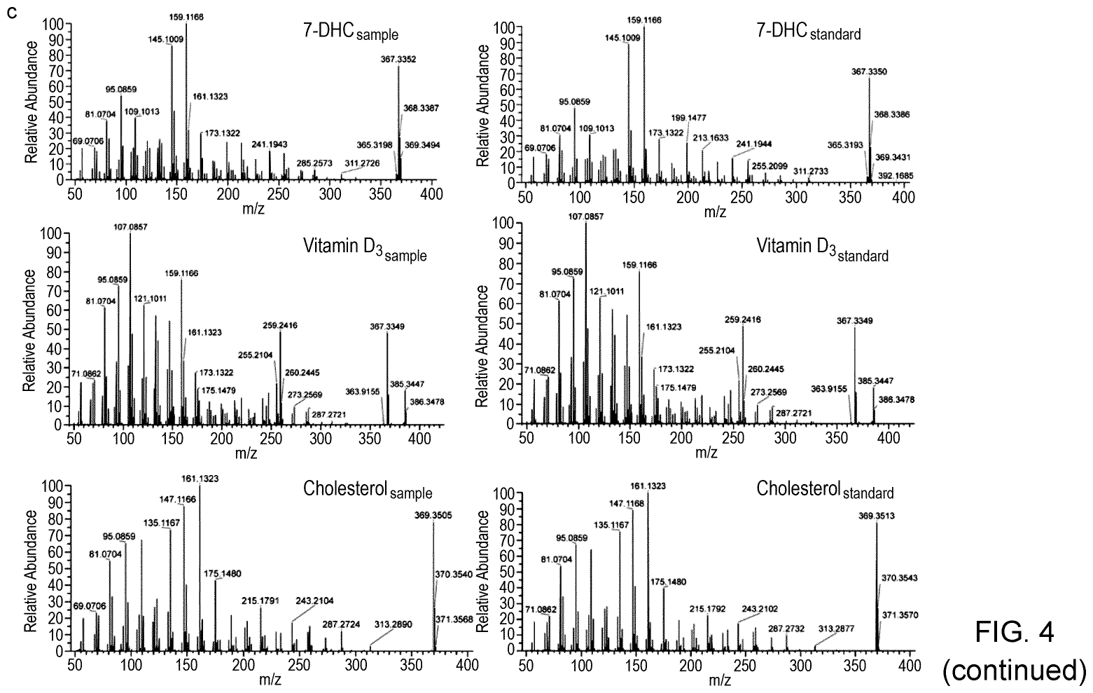

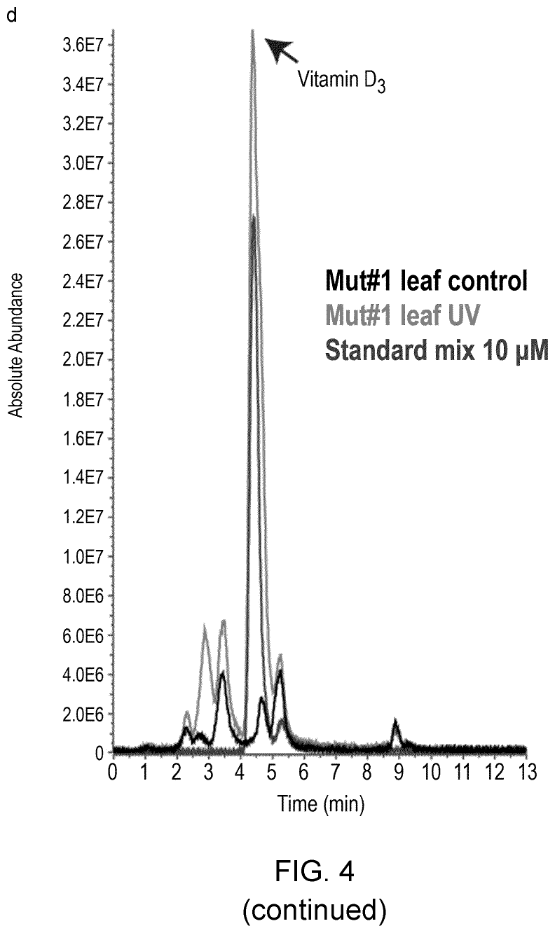

Although provitamin D₃/7-DHC has been identified in tomato leaves it does not normally accumulate in fruit where it serves as an intermediate in the formation of the SGAs; tomatines in green fruit and esculeosides in ripe fruit¹⁶. Recently it has been shown that a duplicate pathway, with specific isoforms of some of the enzymes which are, more generally, responsible for phytosterol and brassinosteroid biosynthesis, produces cholesterol for the formation of SGAs, and is operational in Solanaceous species, including tomato (ref.¹⁷, FIG. 1a). This partial separation of sterol and cholesterol biosynthesis in Solanaceous plants allows metabolic flexibility for the synthesis of important hormones (brassinosteroids) and their more specialised stress chemicals, SGAs, which have fungicidal, antimicrobial, and insecticidal properties. The existence of this ‘duplicate’ pathway for cholesterol and SGA biosynthesis in tomato makes the engineering of provitamin D₃/7-DHC relatively straightforward. A specific isoform of 7-dehydrocholesterol reductase (SI7-DR2) converts provitamin D₃/7-DHC to cholesterol for the synthesis of tomatine in leaves and fruit (FIG. 1a). Consequently, inhibiting the activity of SI7-DR2 in tomato could result in the accumulation of 7-DHC without any impact on phytosterol and brassinosteroid biosynthesis. We have tested the efficacy of blocking SI7-DR2 activity on increasing 7-DHC levels in tomato by creating knockouts of SI7-DR2 using CRISPR/Cas9 genome editing. Two sgRNAs were designed to sequences within the second exon of the gene encoding the SI7-DR2 protein (FIG. 1b), with care being taken to minimise any homology between the sgRNAs and the SI7-DR1 gene. We recovered five independent knockout alleles of the SI7-DR2 gene within the T1 generation, three of which carried identical deletions of 108 bp of exon 2 sequence between the two sgRNAs. Two other knock-out alleles were created by deletion of 2 bp with insertion of 1 bp or insertion of 1 bp only in the second exon, both of which caused frame shifts and predicted premature termination of the SI7-DR2 protein (FIG. 1b). Homozygous knock-out alleles were recovered in the T1 generation, and homozygous knock-out lines lacking the T-DNA carrying the Cas 9 gene and the sgRNA sequences were recovered for 4 of the five lines within the T2 generation.

Five, independently derived, homozygous knock-out alleles of SI7-DR2, four lacking the CRISPR/Cas9 T-DNA, were selected, following segregation, in the T2 generation. No off-target edits of the 7-DR1 gene were detected in these mutant lines. Fruit at different stages of ripening and leaves were analysed for 7-DHC content as well as levels of other phytosterols, cholesterol and SGAs. The sterol and provitamin D₃ profiles of edited and control, wildtype tomato plants were determined using LC-MS^{16,18}.

Loss of SI7-DR2 activity had no effect on the growth or development of the tomato lines (FIG. 3a). This contrasts with the phenotype of the loss-of-function mutation of the equivalent gene involved in phytosterol biosynthesis in Arabidopsis (DWARF5) which is dwarfed because of an inhibition of brassinosteroid biosynthesis¹⁹. The lack of effect of mutations in SI7-DR2 on phytosterol metabolism was confirmed by comparing the levels of stigmasterol, the end product of the phytosterol pathway in tomato, in leaves of wild type and edited lines (FIG. 3b). In wild type, control plants, 7-DHC was detected only in immature green fruit, and was undetectable in ripening and ripe fruit. In contrast, loss of SI7-DR2 activity resulted in substantial increases in provitamin D₃/7-DHC levels in leaves and green fruit (FIGS. 1c and 1d). Levels of 7-DHC were lower in ripe fruit of the SI7-DR2 mutants, but remained high enough that, if converted proportionally to vitamin D₃ (for example by treatment with UV-B light), amounts of vitamin D₃ equivalent to the RDA for supplements (10 μg/day) might be obtained by consuming one or two tomatoes. For the elderly with declining levels of 7-DHC, consuming fruit biofortified with provitamin D₃/7-DHC might address their deficiencies.

MALDI-imaging showed that the increases in provitamin D₃/7-DHC were distributed in both the flesh and peel of tomatoes (FIG. 2a, FIG. 3c). Tomatine and dehydrotomatine are broken down to esculeoside A and B during fruit ripening, meaning that tomatines are reduced to low levels in ripe fruit²⁰. MALDI-imaging of mutant and wild type green fruit showed that α-tomatine was lower in the SI7-DR2 mutant than in controls (FIG. 2a, FIG. 3c,d) and analysis of leaves showed substantially lower levels in the mutants, although a-tomatine was not eliminated (FIG. 2b). A strong reduction in the levels of the SGA, esculeoside A, was also observed in ripe fruit of the mutants compared to the control line (FIG. 2c). These reductions might be considered beneficial because SGAs have been reported to be toxicants/antinutritionals and can be the cause of allergies in consumers. Interestingly the levels of cholesterol in leaves or fruit did not drop compared to controls, and in most mutant lines cholesterol levels were higher than in wild type controls in both fruit (FIG. 2a, FIG. 3e) and leaves (FIG. 2d) This suggested that the block in flux along the

SGA biosynthetic pathway may be compensated by increased flux of intermediates, catalysed by the enzymes of the phytosterol pathway (or at least SI7-DR1), to supplement cholesterol production and to limit reductions in SGA accumulation. However, this does not involve compensatory changes in expression of the genes encoding enzymes in either pathway as shown by qRT-PCR analysis of these genes in leaves of WT and mutant lines (FIG. 4a). The only gene showing consistent changes in transcript levels in the mutants compared to wild type was SIC5-SD1 in the phytosterol pathway, which showed consistently lower transcript levels than in the controls, by about 30%. (FIG. 1a, FIG. 4b).

To determine whether the elevated levels of 7-DHC in SI7-DR2 mutant plants could be converted to vitamin D₃, we treated leaves and sliced fruit to 1 h irradiation by UV-B light as described by Japelt et al.¹⁸. Treatment of leaves was very effective at this conversion resulting in yields of vitamin D₃ of nearly 200 μg per g dry weight (FIG. 2e). Yields of vitamin D₃ from green fruit were lower, reaching about 0.3 μg per g dry weight and in red fruit even lower averaging about 0.2 μg per g dry weight. These lower values in fruit reflected the declining content of 7-DHC precursor in green fruit and red ripe fruit compared to leaves (FIG. 2e). However, considering that a medium-size tomato has a dry weight of about 8-10 g, the levels of vitamin D₃ that can be achieved in a single, SI7-DR2 mutant, green fruit approach 30% and 20% in red fruit of the RDA for the USA and European countries (10-15 μg/day). Clearly, enhancing 7-DHC levels further in ripe fruit would be desirable. Conversion of provitamin D₃ to vitamin D₃ could be enhanced by sun-drying the tomatoes.

The duplicate pathway for cholesterol/SGA biosynthesis exists in other food crops of the family, Solanaceae, including egg plant (Solanum melongena), potato (Solanum tuberosum) and pepper (Capsicum annuum)¹⁷. The close association between cholesterol/SGA biosynthesis, 7-DHC accumulation and photosynthesis in leaves and green fruit of tomato (FIG. 1c-d, FIG. 2b-d, FIG. 3d-e) suggests that knock-outs of SI7-DR2 activity in pepper, where fruit may be green when eaten, might offer an effective additional route to biofortification of plant-based foods in vitamin D₃. In addition, mutations that increase the penetration of UV-B light into fresh fruit, such as the y mutation of tomato, that causes the loss of UV-protecting flavonols from the skin of ‘pink tomatoes’, might also offer increased conversion of provitamin D₃ to vitamin D₃ following UV-B exposure. Such stacking could be achieved by further gene-editing or by introgression²¹. In addition to fortifying fresh fruit with vitamin D₃, the leaves of the SI7-DR2 mutants are rich sources of provitamin D₃ and consequently could provide an important new feed stock using the waste vegetative material from tomato cultivation, for the manufacture of vitamin D₃ supplements from plants, which would be suitable for vegans.

### METHODS

### Plant Materials

Tomato (Solanum lycopersicum) cv. Money Maker plants and SI7-DR 2 knock-out mutants were grown in the greenhouse at the John Innes Centre (Norwich, UK) at an average ambient temperature of 20° C. to 22° C. Supplemental lighting was available to maintain 16 h of light each day when necessary.

### Plasmid Construction

Two specific target sequences (FIG. 1b) in exon 2 of the SI7-DR2 (Solyc06g074090) gene were selected to generate SI7-DR2 knock-out mutants. These were introduced into the sgRNA scaffold by PCR. To make the sgRNA expression cassette, each sgRNA amplicon and a synthesised U6-III promoter (pICSL90001) were cloned into a GoldenGate Level 1 acceptor (pICH47732 and pICH47742). A Level 2 binary vector, pICSL002203, containing the Cas9 expression cassette and kanamycin resistance (nptll) expression cassette, was used as the destination vector to generate the SI7-DR2 CRISPR/Cas9 construct. Plasmids pICSL90001, pICH47732, pICH47742 and pICSL002203 were kindly provided by The Sainsbury Laboratory (TSL) SynBio group (http://synbio.tsl.ac.uk/). sgRNA efficiency was tested by co-transformation of tomato using Agrobacterium rhizogenes (strain ArATCC15834)²². The sequences of exon 2 of SI7-DR2 were amplified by PCR directly from hairy roots with the Phire Plant Direct PCR Master Mix following the manufacturer's instructions (Thermo Fisher Scientific) using primers flanking the sgRNA target sequences SEQ ID NO: 1 (F: TGTTTCACTGGGCTGGTTTAGC and SEQ ID NO: 2R: GAGAAGTCTTTCACCATGTCACGA).

### Tomato Stable Transformation

The SI7-DR2 CRISPR/Cas9 construct was transformed into Agrobacterium tumefaciens (strain AGL1) for stable transformation, which was undertaken using cotyledons as initial explants following a standard transformation protocol as reported previously by Galdon-Armero et al. (2020)²³.

### Screening of SI7-DR2 Knock Out Lines

DNA was isolated from the finely ground powder of leaf tissues using DNeasy® Plant Mini Kits (Qiagen), following the manufacturer's instructions. Five independent SI7-DR2 knock out lines were obtained by genotyping with primers flanking the sgRNA target sequences SEQ ID NO: 3 (F: TGTTTCACTGGGCTGGTTTAGC and SEQ ID NO: 4 R: GAGAAGTCTTTCACCATGTCACGA) and confirmed by sequencing.

### Quantitative Real-Time PCR Analysis

Total RNA was extracted from tomato leaf tissues using the Trizol method (Sigma-Aldrich). DNase I (Roche)-treated RNA was reverse transcribed using SuperScript™ III (Invitrogen). SYBR® Green JumpStart™ Taq ReadyMix™ (Sigma) was used to perform all the RT-qPCR reactions using the X96 Touch™ Real-Time PCR Detection System (Biorad). Data were analysed using CFX Maestro Software. SIActin (Solyc03g078400) was selected as the house-keeping reference gene. The relative expression of genes was caculated by the ΔCt method. Gene-specific primers were designed using NCBI primer BLAST (https://www.ncbi.nlm.nih.gov/tools/primer-blast/), listed in the following table.

| Gene | Oligo | Sequence | Product Size |
|---|---|---|---|
| SlActin (Solyc03g078400) | ACT-q_F SEQ | GGGGGCTATGAATGCACGGT | 110 bp |
| | ID NO: 5 | | |
| | ACT-q_F SEQ | GGCAATGCATCAGGCACCTC | |
| | ID NO: 6 | | |
| SISSR2 (Solyc02g069490) | SSR2_q_F1 | TGTCATTCAAGATCTCCTTGTTCCT | 87 bp |
| | SEQ ID NO: 7 | | |
| | SSR2_q_R1 | CAAATTGGATATACCTCCATCTCGC | |
| | SEQ ID NO: 8 | | |
| SI7-DR1 (Solyc01g009310) | 7-DR1_q_F1 | TTGGGCTTCAGCTTTCCCTT | 88 bp |
| | SEQ ID NO: 9 | | |
| | 7-DR1_q_R1 | AACTCTTGCCTCTGCCTGTC | |
| | SEQ ID NO: 10 | | |
| SISMO4 (Solyc06g005750) | SMO4_q_F1 | GTCCATCATGAGTACGCGACA | 101 bp |
| | SEQ ID NO: 11 | | |
| | SMO4_q_R1 | ATTGCAGGACCAATGACCGT | |
| | SEQ ID NO: 12 | | |
| SISMO3 (Solyc01g091320) | SMO3_q_F1 | TCACAGCGGGTATGAATTTCCA | 106 bp |
| | SEQ ID NO: 13 | | |
| | SMO3_q_R | ACTGTGACAACTTTCCCCAACA | |
| | SEQ ID NO: 14 | | |
| SI3βHSD2 | 3βHSD2_q_F | TAAAGGCCTGCTGACATGCT | 102 bp |
| (Solyc02g081730) | SEQ ID NO: 15 | | |
| | 3βHSD2_q_R | GATTTTCCAGCCCTTGCAGC | |
| | SEQ ID NO: 16 | | |
| SISDR (Solyc01g073640) | SDR_q_F SEQ | GCACGCGGCTAAAGTTATGG | 85 bp |
| | ID NO: 17 | | |
| | SDR_q_R SEQ | GGCCCCAGCTAAAATCCCTT | |
| | ID NO: 18 | | |
| SIC5-SD2 (Solyc02g086180) | C5-SD2_q_F | GGAAGCCTTATGGACGGCTA | 80 bp |
| | SEQ ID NO: 19 | | |
| | C5-SD2_q_R | TGGTATGATAACCGGCACCC | |
| | SEQ ID NO: 20 | | |
| SICPI (Solyc12g098640) | CPI_q_F SEQ | CCCTAGCAAGAGATGGGGTG | 82 bp |
| | ID NO: 21 | | |
| | CP1_q_R SEQ | TGGGACCACAATACCAAGGC | |
| | ID NO: 22 | | |
| SICYP51 (Solyc01g008110) | CYP51_q_F | CGTACAGGCAAGGCAGAGAA | 85 bp |
| | SEQ ID NO: 23 | | |
| | CYP51_q_R | TCTCACCCTCTGTTGTTGGC | |
| | SEQ ID NO: 24 | | |
| SISMO1 (Solyc08g079570) | SMO1_q_F | GGCCAAAGCCAGAGCAATTT | 111 bp |
| | SEQ ID NO: 25 | | |
| | SMO1_q_R | AGCACCCTTTAACTGCTGGA | |
| | SEQ ID NO: 26 | | |
| SISMO2 (Solyc06g076410) | SMO2_q_F | AGAAAGGGCAGGATGGTTGAG | 94 bp |
| | SEQ ID NO: 27 | | |
| | SMO2_q_R | ATACAGCAACAGGCGAGTGA | |
| | SEQ ID NO: 28 | | |
| SI8,7 SI (Solyc06g082980) | 8,7 SI_q_F SEQ | TTGCCTGGTTTTGTTCCCCT | 83 bp |
| | ID NO: 29 | | |
| | 8,7 SI_q_R | AACACCACAACCAGTACGGA | |
| | SEQ ID NO: 30 | | |
| SI7-DR2 (Solyc06g074090) | 7-DR2_q_F | GAGAATGGCCTGCAAGGACTA | 111 bp |
| | SEQ ID NO: 31 | | |
| | 7-DR2_q_R | AGGCAACAAAAGCTGAAGTGT | |
| | SEQ ID NO: 32 | | |
| SIC14-R (Solyc09g009040) | C-14R_q_F | CGGTGTTCAGAGGAGCCAAT | 106 bp |
| | SEQ ID NO: 33 | | |
| | C-14R_q_R | GAAGCAAGCAACTTTCCCCC | |
| | SEQ ID NO: 34 | | |
| SICAS (Solyc04g070980) | CAS_q_F SEQ | GTATTGCTAAAGCTGCCGCC | 86 bp |
| | ID NO: 35 | | |
| | CAS_q_R SEQ | GGTGAAGCAAACTGCCCAAG | |
| | ID NO: 36 | | |
| C5-SD1 (Solyc02g063240) | C5-SD1_q_F | ACTCTCCCCATTTGCTGGTT | 106 bp |
| | SEQ ID NO: 37 | | |
| | C5-SD1_q_R | GTGTGTGGTGAAATGCACAGG | |
| | SEQ ID NO: 38 | | |

### MALDI Imaging Analysis

Cryo-sectioning of fruit was undertaken as described previously by Dong et al. (2020)²⁴. Fresh immature green fruit (about 16 days after anthesis) were flash-frozen in liquid nitrogen and then embedded with M1 embedding matrix (Thermo Scientific) on a flat metal holder on dry ice. The embedded tissues were transferred to a CryoStar NX70 Microtome (Thermo Scientific) and thermally equilibrated at −18° C. for at least 3 h. The tissues were cut into 35 μm thick sections and thaw-mounted on Superfrost Plus slides (Thermo Scientific), followed by vacuum drying in a desiccator.

Optical images were taken using a Canon 5D Mark IV camera with a Canon MP-E 65 mm f/2.8 1-5× Macro Photo lens (Canon Inc, Ōta, Tokyo, Japan) at 1:1 ratio. Image raw files were processed with Capture One photo editing software (Capture One, Frederiksberg, Denmark).

Sections were covered with 2,5-dihydroxybenzoic acid matrix (DHB) using a SunCollect MALDI Sprayer (SunChrome, Friedrichsdorf, Germany) with a DHB solution of 10 mg ml⁻¹ in 80% methanol/0.05% TFA to a density of approx. 3 μg mm^{31 2}.

MALDI imaging was performed with a Synapt G2-Si mass spectrometer with a MALDI source (Waters, Wilmslow, UK) equipped with a 2.5 KHz Nd:YAG laser operated at 355 nm. The slides were fixed in the instrument metal holder and were scanned with a flat-bed scanner (Canon). The images were used to generate pattern files and acquisition methods in the HDImaging software version 1.4 (Waters) with the following parameters: area of a complete section appr. 400 mm², laser beam diameter at low setting (60 μm) with 105 μm step size, resulting in appr. 36k pixels per section, MALDI-MS positive sensitivity mode, m/z 50-1200, scan time 0.5 s, laser repetition rate 1 kHz, laser energy 200. For ion mobility measurements, the same parameters were used in MALDI-HDMS mode with the following additional tune page settings: Trap DC Bias: 45.0, Transfer Wave Velocity (m/s): 315, IMS Wave Height (V): 40.0, Variable Wave Velocity Enabled with linear ramp, Wave Velocity Start (m/s): 1500.0, Wave Velocity End (m/s): 200.0. Red phosphorous clusters were used for instrument calibration and lock mass correction. Total scan time for a complete section was 10-12 h and the lock mass was acquired every 10 min for 2 s.

The MS raw files were processed in HDI1.4 with the following parameters: detection of the 2000 most abundant peaks, m/z window 0.05, MS resolution 10,000, lock mass 526.554 (red phosphorous cluster). The processed data were loaded into HDI1.4 and normalised by total ion content (TIC). Images were generated using the HotMetal2 colour scale and exported as png image files. For comparison and generation of mean spectra, the MS raw data were transformed into imzml and analysed using the Scils Lab MVS software version 2021c Premium 3D (SCiLS, Bruker Daltonik GmbH, Bremen, Germany).

Compounds of interest, 7-dehydrocholesterol, cholesterol and α-tomatine were identified by comparing the drift time and mass of authentic standards analysed on the same instrument. The masses detected for 7-dehydrocholesterol, vitamin D₃ and cholesterol during MALDI are listed in the following table. It has been reported that cholesterol is susceptible to laser-induced oxidisation during MALDI-TOF mass spectrometry²⁵, and 7-dehydrocholesterol has an even higher tendency for non-enzymatic autoxidation^{26,27}. Among the peaks of standards generated during MALDI, taking into account their specificity and relative abundance, 367.33, 365.32 and 363.31 were selected as representative masses for 7-dehydrocholesterol, 369.35 and 1034.55 were selected as representative masses for cholesterol and α-tomatine, respectively.

| Standards spotted side by | | | | | | | | |
|---|---|---|---|---|---|---|---|---|
| side on WT tissue section | | | | Standards spotted on | | | | |
| | | | colocal- | glass slides separately | | | | |
| | normalised | drift time | ised | cholcal | | | 7dh | |
| m/z | intensity | (dt) (bins) | with | m/z | intensity | dt bins | m/z | intensity |
| 360.3638 | 0.06069 | 12.30 | 7dh and | 360.3530 | 1708 | 12.27 | | |
| | | | chol* | | | | | |
| 361.3667 | 0.01652 | 12.28 | | | | | 361.2889 | 2285 |
| 363.3063 | 0.06303 | 11.00 | 7dh | 363.3050 | 682 | 10.97 | 363.3047 | 17699 |
| | | | | | | | 364.3088 | 6183 |
| 365.3210 | 0.0324 | 10.99 | 7dh | 365.3204 | 4144 | 11.08 | 365.3192 | 15508 |
| 366.3024 | 0.0083 | 11.25 | 7dh | 366.3253 | 1884 | 11.24 | 366.3237 | 6149 |
| | | | | 367.3357 | 11782 | 11.23 | 367.3301 | 7997 |
| | | | | 368.3391 | 3171 | 11.24 | 368.3340 | 2587 |
| 369.3471 | 0.2623 | 11.74 | chol | 369.3192 | 1713 | 11.39 | 369.3162 | 4587 |
| 370.2974 | 0.01066 | 11.27 | 7dh and | | | | | |
| | | | chol | | | | | |
| | | | | | | | 373.3100 | 1862 |
| 376.3227 | 0.02078 | 11.56 | cholcal | | | | | |
| 377.3254 | 0.01356 | 11.55 | cholcal | | | | | |
| | | | | | | | 379.2997 | 6491 |
| 380.3334 | 0.25962 | 10.42 | | | | | 380.3202 | 5738 |
| 381.3328 | 0.08153 | 10.68 | | 381.3138 | 1098 | 11.48 | 381.3159 | 13342 |
| 382.3397 | 0.0398 | 10.80 | | 382.3255 | 906 | 11.69 | 382.3328 | 7426 |
| | | | | 383.3272 | 3248 | 11.71 | 383.3342 | 4302 |
| | | | | 384.3326 | 1615 | 11.86 | 384.3433 | 3929 |
| | | | | 385.3326 | 2458 | 11.69 | | |
| 388.3957 | 0.04975 | 13.18 | 7dh and | 388.3934 | 931 | 13.26 | | |
| | | | chol | | | | | |
| 389.3983 | 0.03727 | 13.17 | 7dh and | | | | | |
| | | | chol | | | | | |

| Standards spotted on | | | | m/z Observed in mutants with | | | |
|---|---|---|---|---|---|---|---|
| glass slides separately | | | | same typical localisation pattern | | | |
| 7dh | chol | | | Mut#1 | | Mut#5 | |
| dt bins | m/z | intensity | dt bins | m/z | dt bins | m/z | dt bins |
| | 360.3619 | 607 | 12.26 | | | | |
| 10.88 | | | | | | | |
| 10.92 | | | | 363.3043 | 10.93 | 363.3058 | 11.03 |
| 10.95 | | | | | | | |
| 10.92 | | | | 365.3190 | 10.93 | 365.3182 | 11.01 |
| 11.01 | | | | | | | |
| 11.14 | 367.3384 | 1366 | 11.22 | 367.3336 | 11.27 | 367.3242 | 11.43 |
| 11.18 | 368.3438 | 1037 | 11.39 | | | | |
| 11.04 | 369.3546 | 14248 | 11.54 | 369.3381 | 11.78 | 369.3350 | 11.82 |
| | 370.3576 | 3924 | 11.55 | | | | |
| 10.91 | | | | | | | |
| 11.20 | | | | | | | |
| 11.33 | | | | | | | |
| 11.20 | | | | 381.3266 | 10.79 | 381.3273 | 11.00 |
| 11.07 | | | | 382.3340 | 10.77 | 382.3319 | 11.21 |
| 11.20 | | | | 383.3567 | 11.62 | 383.3497 | 11.78 |
| 11.47 | | | | | | | |
| | 388.3950 | 1023 | 13.19 | | | | |

*cholcal: Cholecalciferol, vitamin D₃; 7dh: 7-dehydrocholesterol (7-DHC); chol: Cholesterol.

### Sterol Analysis

The methods for sterol extraction and analysis were modified from Jäpelt et al. (2011)¹⁶. Freeze-dried material (about 20 mg) was weighed into a 2 mL Eppendorf tube and mixed with 100 μL 60% potassium hydroxide (Sigma-Aldrich), 500 μL 96% ethanol (Sigma-Aldrich) and 300 μL 15% ascorbic acid (Sigma-Aldrich). The tubes were shaken for approximately 18 h at 22° C. in a thermoshaker (Eppendorf). 20% ethyl acetate in pentane (v/v) (750 μL) was added and shaken for 30 min on a flat shaker, followed by centrifugation at 2000×g for 5 min at room temperature. The organic layer was transferred into a new 2 ml Eppendorf tube. The extraction steps were repeated, twice. Total extracts were washed with 500 μL of 0.1 mol L⁻¹ hydrochloric acid by inverting tubes 30 times to completely remove the alkali. The upper layer was transferred into a 2 mL Eppendorf tube following centrifugation at 1000×g for 2 min. Total extracts were evaporated to dryness using a Genevac EZ-2 Elite Evaporator with the programme of ‘Very Low BP Mix’. The residue was finally redissolved in 200 μL methanol and filtered through 0.22 μm nylon Corning® Costar® Spin-X® tube filter (Sigma-Aldrich). The samples were stored at −80° C. until analysis.

Sterol compounds were identified and quantified by comparing the retention time and mass spectrometry spectra of authentic standards analysed on the same instrument: 7-dehydrocholesterol, vitamin D₃, cholesterol, stigmasterol (Sigma-Aldrich). Liquid chromatographic analysis was performed on a Dionex UltimMate (Thermo Fisher Scientific) equipped with a thermostated column compartment. The chromatographic separation was done on a 50×2.1 mm 2.6μ Kinetex F5 column (Phenomenex) at a flow-rate of 0.6 mL min⁻¹. Solvents were was 0.2% formic acid and 25% acetonitrile in Milli-Q water (v/v) (A) versus 100% methanol (B). The gradient program was as follows: 60% B for 0.5 min, a linear gradient to 85% B for 7 min, a linear gradient to 100% B for 0.5 min, isocratic elution for 1 min and 0.5 min linear gradient back to 60% B and re-equilibration for 3.5 min giving a total run time of 13 min. The column was maintained at 40° C. 5 μL samples were injected. Mass spectrometry was performed using a Q Exactive Orbitrap Mass Spectrometer (Thermo Scientific) with an atmospheric pressure chemical ionisation (APCI) source. The MS was set up to collect full scans at 70,000 resolution from m/z 180-2000, and data dependent MS2 of the top 4 ions, at an isolation width of m/z 4.0, 30% normalised collision energy. These ions were then ignored for 5 sec in favour of the next most abundant ion; isotope peaks were also ignored. Data-dependent MS2 analysis was at 17,500 resolution, maximum ion time of 50 msec, automatic gain control target of 1×105 ions. MS scans had a maximum ion time of 50 msec, and automatic gain control target of 3×106 ions. Spray chamber conditions were 231° C. capillary temperature, 21.25 units sheath gas, 5 units aux gas, no spare gas, 4 μA current, 363° C. probe heater temperature, and 50V S-lens RF. Xcalibur software (version 4.3, Thermo Scientific) was used for instrument control and data acquisition.

### SGA Analysis

SGA extraction was performed as described by Itkin et al. (2011)²⁸. Briefly, 20 mg of freeze-dried sample (leaf or fruit) were sonicated in 1 mL 100% methanol, incubated on ice for 1 h and centrifuged at 4000 rpm for 10 min. The supernatants were collected, centrifuged at 4000 rpm for 3 min and filtered with 0.22μ filter. The sample extracts were stored at −80° C. until the analysis. α-tomatine was identified and quantified by comparing the retention time and mass spectrometry spectrum to the authentic standard (Sigma-Aldrich). Esculeoside A was identified based on its mass spectrometry spectrum compared with previously published spectra and relative retention time (Itkin et al. 2011).

The chromatographic separation was done on a 50×2.1 mm 2.6μ Kinetex EVO C18 column (Phenomenex) at a flow-rate of 0.6 mL min⁻¹. Solvents were 0.1% formic acid in Milli-Q water (v/v) (A) versus 100% acetonitrile (B). The gradient program was as follows: a linear gradient from 2% B to 40% B for 4 min, and then a linear gradient to 95% B for 2 min, isocratic elution for 1 min and 0.1 min linear gradient back to 2% B and re-equilibration for 2.1 min giving a total run time of 9.2 min. Mass spectrometry was performed using a Q Exactive Orbitrap Mass Spectrometer (Thermo Scientific) with an electrospray ionization (ESI) source. All other settings were the same as those described above for sterol analysis.

### UV Treatment

Fruits were cut into 1 mm slices before UV exposure. Leaf or fruit tissues were exposed to UV-B light (3.2 mW cm^{31 2}) for 1 h at 20 cm below the inverted short wavelength transilluminator. Samples were immediately frozen in liquid nitrogen after treatment for following analysis.

### Statistical Analysis

All the experiments in this paper were repeated at least three times independently and results from representative data sets are presented. All numerical values are presented as means±s.e.m. GraphPad Prism (version 9.2.0) was used for the statistical analysis. Statistical differences between wild type and mutants were conducted using two-tailed Student's t-tests.

### REFERENCES

1. McCollum, E.V., Simmonds, N., Becker, J.E. & Shipley P.G. (1922) J Biol Chem; 53:293-312.
2. Bikle, D. & Christakos, S., (2020). Nature Reviews Endocrinology, 16:234-252.
3. Hossain, S., et al., 2019. Clinical Nutrition ESPEN, 30, 170-184.
4. Garland C. & Garland F. (1980) Int J. Epidemiol. 9:227-231.
5. Zhou, Z., Zhou, R., Zhang, Z. & Li, K., (2019). Medical Science Monitor: International Medical Journal of Experimental and Clinical Research, 25, p.666.
6. Anglin, R.E., Samaan, Z., Walter, S.D. & McDonald, S.D., (2013). The British Journal of Psychiatry, 202:100-107.
7. Llewellyn, D.J., et al., 2010. Archives of Internal Medicine, 170:1135-1141.
8. Sommer, I., et al., (2017). BMC Geriatrics, 17:1-13.
9. Pizzini, A., et al., (2020). Nutrients, 12:2775.
10. Holick, M.F., et al., (1980). Science, 210:203-205.
11. Jäpelt, R.B. & Jakobsen, J., (2013). Frontiers in Plant Science, 4: p. 136.
12. van Schoor, N. & Lips, P., (2018). Worldwide vitamin D status. Vitamin D, pp. 15-40.
13. Black, L.J., Lucas, R.M., Sherriff, J.L., Björn, L.O. & Bornman, J.F., (2017). Nutrients, 9: p.136.
14. Armas, L.A., Hollis, B.W. & Heaney, R.P., (2004). The Journal of Clinical Endocrinology & Metabolism, 89:5387-5391.
15. Heaney, R.P., Recker, R.R., Grote, J., Horst, R.L. & Armas, L.A., (2011). The Journal of Clinical Endocrinology & Metabolism, 96: E447-E452.
16. Jäpelt, R.B., Silvestro, D., Smedsgaard, J., Jensen, P.E. & Jakobsen, J., (2011). Food Chem., 129:217-225
17. Sonawane, P.D., et al., (2016). Nature Plants, 3:1-13.
Boland, R., Skliar, M., Curino, A. & Milanesi, L., (2003). Plant Science, 164:357-369.
18. Jäpelt, R.B., Silvestro, D., Smedsgaard, J., Jensen, P.E. & Jakobsen, J., (2013). Food Chemistry, 138:1206-1211.
19. Choe, S., et al., (2000). The Plant Journal, 21: .431-443.
20. Kozukue, N., Han, J.S., Lee, K.R. & Friedman, M., (2004). J. Agric. Food Chem., 52:2079-2083
21. Ballester, A.R., et al., (2010). Plant Physiology, 152: .71-84.
22. Ron, Mily et al., (2014). Plant physiology, vol. 166,2 (2014): 455-69.
23. Galdon-Armero, J., Arce-Rodriguez, L., Downie, M., Li, J., & Martin, C. (2020). The Plant cell, 32(5), 1414-1433.
24. Dong, Y., et al., (2020). The new phytologist, 228(6), 1986-2002.
25. McAvey, K. M., et al., Journal of the American Society for Mass Spectrometry, 22(4), 659-669.
26. Xu, L., Davis, T. A., & Porter, N. A. (2009). Journal of the American Chemical Society, 131(36), 13037-13044.
27. Xu, L., Korade, Z., & Porter, N. A. (2010). Journal of the American Chemical Society, 132(7), 2222-2232.
28. Itkin, M. et al. The Plant Cell, 23, 4507-4525 (2011).

1. A method of improving provitamin D₃ levels in a plant, the method comprising reducing activity of 7-dehydrocholesterol reductase (7-DR) in the plant.

2. The method of claim 1 wherein the method comprises introducing one or more mutations reducing the activity of the enzyme encoded by the 7-DR gene in the plant.

3. The method of claim 2 wherein the plant genome comprises a duplication of the 7-DR gene, and the one or more mutations are introduced into the 7-DR2 gene.

4. The method of claim 2 or 3, further comprising breeding mutant plants to obtain a plant homozygous for the mutation.

5. The method of claim 1, wherein a post-transcriptional technique is used to reduce or abolish enzyme activity.

6. The method of any preceding claim further comprising exposing the plant or a part of the plant to UV-B radiation.

7. The method of any preceding claim wherein the plant carries a mutation which increases the penetration of UV-B light into fruit.

8. The method of any preceding claim further comprising processing the plant or a part of the plant to obtain 7-DHC and/or vitamin D₃.

9. A genetically altered plant, part thereof or plant cell, wherein the plant, part thereof or plant cell has reduced activity of 7-dehydrocholesterol reductase.

10. The plant, part thereof or plant cell of claim 9, comprising a loss of function mutation in the 7-DR gene.

11. The plant, part thereof or plant cell of claim 10 wherein the loss of function mutation is in the 7-DR2 gene.

12. The method of any of claims 1 to 8, or the plant, part thereof or plant cell of any of claims 9-11, wherein the plant is a member of the Solanaceae, more preferably Solanum spp, and most preferably selected from tomato (Solanum lycopersicum), potato (Solanum tuberosum), eggplant (Solanum melongena).

13. A food product produced from a plant or plant part of any of claims 9 to 11.
